(19) United States
(12) Patent Application Publication
(10) Pub. No.: US 2026/0076879 A1
(43) Pub. Date: Mar. 19, 2026

(54) ALUMINUM-FREE, FATTY-ACID-COATED INORGANIC POWDER AND METHOD FOR PREPARING SAME

(71) Applicant: SUNJIN BEAUTY SCIENCE CO., LTD, Seoul (KR)

(72) Inventors: Sung Ho LEE, Seoul (KR); Yeong Gi KIM, Seocheon-gun (KR); Won Jun JANG, Gunsan-si (KR)

(21) Appl. No.: 19/107,152

(22) PCT Filed: Sep. 4, 2023
(86) PCT No.: PCT/KR2023/013190

§ 371 Date: Feb. 27, 2025

(30) Foreign Application Priority Data
Sep. 6, 2022 (KR) 10-2022-0112634
Mar. 24, 2023 (KR) 10-2023-0038698
May 24, 2023 (KR) 10-2023-0067263

(51) Int. Cl. A61K 8/02; A61K 8/25; A61K 8/27; A61K 8/29; A61K 8/36; A61K 8/365; A61Q 19/00

(52) U.S. Cl. CPC A61K 8/0241; A61K 8/25; A61K 8/27; A61K 8/29; A61K 8/361; A61K 8/365; A61Q 19/00; A61K 2800/30; A61K 2800/43; A61K 2800/621; A61K 2800/622; A61K 2800/651

(57) ABSTRACT

The present invention relates to a fatty acid-coated inorganic powders for cosmetics, comprising a zinc-fatty acid soap coating layer on the surface of the inorganic powders through dry coating, and a method of preparing the same. In particular, by adding zinc oxide to the inorganic powder, adding fatty acid thereto, and reacting the same by heating so that zinc-fatty acid soap is coated on the inorganic powder, even if unreacted substances remain during production, both zinc oxide and fatty acid are substances that are widely used in cosmetics and are safe for the skin, and since they do not use aluminum, they can improve the negative image of causing Alzheimer's disease, and further, since the inorganic powder coated with zinc-fatty acid soap is manufactured by a dry method, it is possible to prevent environmental pollution by not using water, and also to save energy and shorten the manufacturing time.

### TECHNICAL FIELD

The present invention relates to a fatty acid-coated inorganic powder that does not contain aluminum and a method of preparing the same, and in particular, the present invention relates to a fatty acid-coated inorganic powder and a method of preparing the same, wherein zinc oxide is added to an inorganic powder, fatty acid is added thereto, and a heating reaction is performed to coat the inorganic powder with zinc-fatty acid soap. Even if the unreacted zinc oxide and fatty acid remain during manufacturing, both of these are widely used in cosmetics and are safe for the skin, and since aluminum is not used, the negative image of causing Alzheimer's disease can be improved. In addition, since the inorganic powder coated with zinc-fatty acid soap is produced by a dry method, not only can environmental pollution be prevented because water is not used, but also energy can be saved and the production time can be shortened.

### BACKGROUND ART

The content described below merely provides background information related to the present invention and does not constitute prior art.

Inorganic powders are widely used in cosmetics such as makeup products and UV blocking products, for their color, coverage, and UV blocking effects.

However, most inorganic powders used in cosmetics have poor oil dispersibility, and the catalytic action of the metal ions of the inorganic powders can promote oxidation reactions of ingredients used in cosmetics, thereby lowering the quality of the cosmetics. Therefore, the surface of the inorganic powder is coated with a material having lipophilic properties to compensate for these shortcomings.

As a method of surface treatment of inorganic powders, methods such as coating inorganic powders with silica or alumina to change their properties or coating organic polymers have been developed and commercialized. In particular, the surface of the cosmetics is treated with oil to increase its dispersibility in oil and prevent stains or easy erasure by moisture such as sweat.

In particular, the surface is treated with oil to increase the dispersibility of cosmetics in oil and to prevent them from being stained or easily removed by moisture such as sweat.

Among these oil-based treatment methods, the method of surface-treating alkyl silanes such as trimethoxyisobutylsilane, trimethoxyoctylsilane, and triethoxycaprylicsilane, and the method of coating metal-fatty acid soap are widely used. Alkyl silanes are widely used as oil-based materials for treating the surface of inorganic powders due to their high reactivity, but there is a growing trend to not use silane compounds in cosmetics, and due to concerns about its safety for skin, powders treated with fatty acids are preferred over alkylsilane treatment.

The conventional method of coating metal-fatty acid soap on inorganic powder consists dispersing the inorganic powder in water, adding an alkali-fatty acid soap thereto, and then heating to about 60 to 80° C., and then adding aluminum chloride, aluminum sulfate, aluminum phosphate, or zinc chloride, zinc sulfate, zinc phosphate, etc. to generate a metal-fatty acid soap, and allowing the generated metal soap to be adsorbed on the inorganic powder. Afterwards, it is cooled, washed, and dehydrated to obtain inorganic powder coated with metal soap. And then it is dried, crushed, and screened to manufacture inorganic powder coated with metal soap, which is the most widely used manufacturing method.

However, this wet manufacturing method of metal soap-coated inorganic powder has the advantage of being able to generate a metal-fatty acid soap film on the surface of the inorganic powder and can easily react, but it uses a lot of water, which can cause environmental problems such as wastewater, and also consumes a lot of energy in the process of heating and cooling the water and drying the manufactured metal soap-coated inorganic powder, and the manufacturing time is relatively long.

To overcome these shortcomings, a dry metal-fatty acid soap coating method that does not use water has been investigated. However, most inorganic powders are acidic in nature and do not have a binding force with fatty acids, so it was confirmed that aluminum hydroxide, which has relatively high reactivity, was mixed with inorganic powder, fatty acids were added to it, and then heated to coat the surface of the inorganic powder with aluminum fatty acid soap.

However, when aluminum hydroxide is used, unreacted aluminum ions may exist, and there is a research report that this aluminum binds to amyloid and beta protein in the brain in an aggregated form and causes Alzheimer's disease, so it is not desirable to use aluminum hydroxide to coat aluminum fatty acid soap on the inorganic powders. Similarly, when zinc hydroxide was used for the reaction, the coating did not occur well on the inorganic powder like aluminum hydroxide. In addition, when zinc hydroxide is used, unreacted zinc ions may exist after the reaction, and this may act as a catalyst to promote oxidation of substances used in cosmetics, and may also cause irritation to the skin, so a dry coating method of metal soap on inorganic powder through another method was urgently required.

Another method is to directly coat the inorganic powder by mixing zinc-fatty acid. However, it was confirmed that even this method did not coat the inorganic powders with zinc fatty acid to a satisfactory level.

Therefore, in the present invention, in addition to the method of directly using zinc hydroxide or zinc-fatty acid soap, it was confirmed that a zinc-fatty acid coating was formed by reacting with fatty acids on the surface of zinc oxide, and this led to the development of a method for dry coating zinc-fatty acid soap onto an inorganic powder using the zinc oxide.

### PRIOR ART LITERATURE

### Patent Literature

1. U.S. Pat. No. 4,648,908 (Mar. 10, 1987)
2. Korean Patent Publication No. 10-2007-0726989 (Jun. 14, 2007)
3. Korean Patent Publication No. 10-2012-0053258 (May 25, 2012)

### DISCLOSURE

### Technical Problem

The technical problem to be achieved by the present invention is to solve the conventional problems, and the purpose of the present invention is to provide an inorganic powder coated with fatty acid that does not contain aluminum and a method of preparing the same, in which after adding zinc oxide to the inorganic powder, fatty acid is added to it and the reaction is heated to coat the inorganic powder with zinc-fatty acid soap. Since both zinc oxide and fatty acid are substances that are widely used in cosmetics and are safe for the skin, so it is safe for the skin and can improve the negative image that aluminum causes Alzheimer's disease by not using it. Furthermore, since the inorganic powder coated with zinc-fatty acid soap is manufactured by a dry method, it is possible to prevent environmental pollution by not using water, and also to save energy and shorten the manufacturing time.

### Technical Solution

The present invention provides Inorganic powders for cosmetics coated with fatty acids, comprising: a zinc-fatty acid soap coating layer on the surface of the inorganic powders through dry coating.

Preferably, the inorganic powders may be at least one selected from the group consisting of titanium dioxide, mica, talc, kaolin, sericite, yellow iron oxide, red iron oxide, black iron oxide, brown iron oxide, chromium oxide, silica, and titanium dioxide mica.

Specifically, the fatty acid may be at least one selected from the group consisting of caprylic acid, capric acid, lauric acid, myristic acid, palmitic acid, stearic acid, isostearic acid, hydroxystearic acid, oleic acid, linoleic acid, and behenic acid.

Preferably, the zinc-fatty acid soap coating layer is included in an amount of 2 to 50 parts by weight based on 100 parts by weight of the inorganic powders.

In addition, when aluminum is included in the form of an impurity, aluminum is included in an amount of 20 ppm or less.

Also, the present invention provides a method of preparing inorganic powders for cosmetics coated with zinc-fatty acid soap, comprising: a first step of homogeneously mixing an inorganic powder and zinc oxide; a second step of homogeneously stirring mixture of the first step by adding fatty acid; a third step of heating and reacting stirred material of the second step; and a fourth step of cooling and pulverizing reactant of the third step.

Preferably, in the first step, 1 to 20 parts by weight of zinc oxide is included relative to 100 parts by weight of the inorganic powder.

Preferably, in the second step, 50 to 500 parts by weight of fatty acid is included relative to 100 parts by weight of the zinc oxide.

Preferably, in the third step, heating is performed at 100 to 150° C.

In addition, even when aluminum is included in the form of an impurity, aluminum is included in an amount of 20 ppm or less.

The present invention also provides a cosmetic comprising inorganic powders coated with the zinc-fatty acid soap described above.

### Advantageous Effects

According to the present invention, by adding zinc oxide to an inorganic powder and adding fatty acid thereto and reacting by heating, the inorganic powder is coated with zinc-fatty acid soap, so that even if unreacted substances remain during manufacturing, both zinc oxide and fatty acid are substances that are widely used in cosmetics and are safe for the skin, and since aluminum is not used, the negative image of it causing Alzheimer's disease can be improved, and further, since the fatty acid-coated inorganic powder is manufactured by a dry method, water is not used, so environmental pollution can be prevented, energy can be saved, and manufacturing time can be shortened.

### DESCRIPTION OF DRAWINGS

FIG. 1 is a photograph of the present invention confirming that the zinc oxide is coated with fatty acid, showing that the coated fatty acid is not separated when boiled in water for 10 minutes.

FIG. 2 shows an energy dispersive spectroscopy (EDS) photograph of a scanning electron microscope (SEM) of a powder according to an example of the present invention.

FIG. 3 is a photograph of titanium dioxide insufficiently coated with fatty acids when boiled in water for 1 minute according to a comparative example of the present invention.

FIG. 4 is a photograph of a comparative example of the present invention, in which stearic acid was coated on titanium dioxide using aluminum hydroxide and then boiled in water for 30 minutes.

FIG. 5 is a photograph of powder according to an example of the present invention when boiled in water for 30 minutes.

FIG. 6 is a result of analysis using WD-XRF (Wavelength Dispersion X-ray Fluorescence Spectrometer) to confirm the presence of aluminum components in titanium dioxide powder including a zinc-fatty acid coating layer according to an example of the present invention and those of competitors (3 companies).

FIG. 7 is a result of analyzing the titanium dioxide powder including a zinc-fatty acid coating layer according to an example of the present invention using FT-IR.

FIG. 8 is a photograph showing that zinc-fatty acid is not separated when zinc-fatty acid is coated on silica and is heated in water for 10 minutes according to an example of the present invention.

FIG. 9 is a photograph of an example of the present invention, in which zinc hydroxide was used instead of zinc oxide to coat silica with zinc fatty acid and then placed in water and boiled for 3 minutes. In the case of zinc oxide, it can be confirmed in Example 2 that zinc-fatty acid soap is coated, but when zinc hydroxide is used instead of zinc oxide, it can be confirmed that zinc-fatty acid soap is not coated.

FIG. 10 is a photograph of a zinc-fatty acid soap coated on a sericite using zinc oxide and stearic acid and then placed in water and boiled for 10 minutes according to an example of the present invention. It can be confirmed that the zinc-fatty acid is coated on the sericite.

FIG. 11 is a photograph of yellow iron oxide coated with zinc-fatty acid soap after boiling it in water for 10 minutes according to an example of the present invention. It can be confirmed that it is coated.

FIG. 12 is a photograph of a zinc-fatty acid soap coated red iron oxide after boiling it in water for 10 minutes according to an example of the present invention. It can be confirmed that it is coated.

### BEST MODE

In order to fully understand the configuration and effect of the present invention, preferred embodiments of the present invention will be described with reference to the attached drawings. However, the present invention is not limited to the embodiments disclosed below, but can be implemented in various forms and can be changed in various ways.

The terminology used herein is intended to describe embodiments and is not intended to limit the present invention. In the present invention, the singular includes the plural unless specifically stated otherwise. The terms “comprise(s)” and/or “comprising” as used in the present invention do not exclude the presence or addition of one or more other components.

The present invention relates to an inorganic powder having a zinc-fatty acid soap coated on the surface of the inorganic powder, and in order to solve the disadvantages of environmental pollution, large energy consumption, and long manufacturing time in the wet method of the conventional metal-fatty acid coating method, a method of manufacturing using a dry method was devised.

It was confirmed that an inorganic powder coated with aluminum-fatty acid was manufactured using highly reactive aluminum hydroxide first by the dry method.

However, aluminum was reported to cause Alzheimer's disease, so it needed to be replaced, and in the case of zinc hydroxide, the coating did not occur properly, and it was also found that coating of zinc-fatty acid soap by a dry method by directly mixing inorganic powder and zinc-fatty acid soap did not form a uniform coating film. Accordingly, as a result of using zinc oxide under specific conditions, it was confirmed that a zinc-fatty acid coating layer with significantly excellent stability was formed.

Therefore, the inorganic powder of the present invention is an inorganic powder for cosmetics coated with zinc-fatty acid soap, and is characterized by comprising a zinc-fatty acid coating layer directly formed on the surface of the inorganic powder through dry coating.

The inorganic powder of the present invention is characterized in that even when aluminum is contained in the form of impurities, aluminum is contained in an amount of 20 ppm or less.

The inorganic powder for cosmetics coated with zinc-fatty acid soap can be manufactured through a method of preparing inorganic powders coated with zinc-fatty acid soap, comprising:
a first step of homogeneously mixing inorganic powder and zinc oxide;
a second step of homogeneously stirring the mixture of the first step by adding fatty acid;
a third step of heating and reacting the stirred material of the second step; and
a fourth step of cooling and pulverizing the reactant of the third step;

The method for preparing inorganic powders coated with zinc-fatty acid soap of the present invention is characterized in that it does not use aluminum hydroxide, which is highly reactive with fatty acid, and does not directly use zinc-fatty acid soap, but rather adds fatty acid to a mixture of inorganic powder and zinc oxide and reacts the mixture.

The inorganic powder of the first step may be a known inorganic powder used in cosmetics, and preferably, the inorganic powder may be at least one selected from the group consisting of titanium dioxide, mica, talc, kaolin, sericite, yellow iron oxide, red iron oxide, black iron oxide, brown iron oxide, silica, titanium dioxide mica, and zinc oxide.

In the above, mica, talc, kaolin, sericite, etc. are filler pigments, yellow iron oxide, red iron oxide, black iron oxide, brown iron oxide, etc. are colored pigments, titanium dioxide, silica, zinc oxide, etc. are white pigments, and titanium dioxide mica is a pearlescent pigment.

In addition, in the first step, the amount of zinc oxide may be 1 to 20 parts by weight per 100 parts by weight of the inorganic powders, and preferably 2 to 10 parts by weight. If the amount of zinc oxide is too small beyond the above range, zinc-fatty acid soap is not sufficiently formed, resulting in insufficient coating. If the amount of zinc oxide is too large, unreacted zinc oxide remains in large quantities, which may change the properties of the coated inorganic powder.

The fatty acid used in the second step may preferably be at least one selected from the group consisting of caprylic acid, capric acid, lauric acid, myristic acid, palmitic acid, stearic acid, isostearic acid, hydroxystearic acid, oleic acid, linoleic acid, and behenic acid.

The amount of the fatty acid may be 50 to 500 parts by weight based on 100 parts by weight of the zinc oxide used, and preferably 100 to 200 parts by weight. If the amount of the fatty acid is too small beyond the above range, zinc-fatty acid soap is not sufficiently formed. In addition, if the amount of the fatty acid is too large, unreacted fatty acids remain, resulting in poor usability and adversely affecting the properties of the final product.

In the third step, it is preferable to react at a temperature of 100 to 150° C. More preferably, the reaction is performed at 110 to 130° C. If the reaction temperature is lower than 100° C., a dehydration reaction occurs when the fatty acid and zinc oxide react to form metal soap, so water is not sufficiently removed, resulting in insufficient reaction. In addition, since the formed zinc-fatty acid soap dissolves at about 110 to 120° C., it is better to react at a temperature higher than 110° C. for uniform coating. However, if the temperature is too high, the reaction speed increases, but there is a risk that the fatty acid may change color or smell.

In the fourth step, when the powder after the reaction is cooled to room temperature, some of the powder is agglomerated, so it is pulverized and sieved through a 100 to 300 mesh sieve to manufacture an inorganic powder coated with zinc-fatty acid soap. If necessary, a drying process may be further included.

In the method of preparing inorganic powders coated with zinc-fatty acid soap of the present invention, the first step and the second step may be performed simultaneously, or the second step and the third step may be performed simultaneously. Preferably, the first step, the second step, and the third step are performed sequentially.

The method of preparing inorganic powders coated with zinc-fatty acid soap according to the present invention is manufactured using a dry manufacturing method, so that water is not used, and thus environmental pollution can be prevented, energy can be saved, and manufacturing time can be shortened.

In addition, in some cases, even if impurities including aluminum components are included, since the aluminum content is included in a trace amount of 20 ppm or less, the negative image that aluminum causes Alzheimer's disease can be improved.

The inorganic powder for cosmetics coated with zinc-fatty acid soap according to the present invention does not contain aluminum or zinc ions, and it is preferable that the zinc-fatty acid soap coating layer is included in an amount of 2 to 50 parts by weight per 100 parts by weight of the inorganic powder. In this case, the inorganic powder coated with zinc-fatty acid has excellent water resistance in cosmetics, and can produce cosmetics with a good feeling of use.

The inorganic powder for cosmetics coated with zinc-fatty acid soap according to the present invention has a uniform zinc-fatty acid coating layer directly adjacent to the surface of the inorganic powder as observed by scanning electron microscope (SEM).

In addition, when added to water and boiled for 30 minutes or more, the fatty acid and inorganic powder were not separated, indicating that the fatty acid is bound and attached to the surface of the inorganic powder.

Therefore, the inorganic powder coated with zinc-fatty acid soap according to the present invention not only has excellent water resistance, but can also improve the usability and stability of cosmetics when applied to cosmetics. In addition, in some cases, even when included as an impurity including aluminum components, the possibility of causing Alzheimer's disease due to aluminum can be eliminated as the aluminum content is included in an extremely small amount of 20 ppm or less.

Hereinafter, the present invention will be described in detail through examples. However, the following examples are only for illustrating the present invention, and the present invention is not limited to the following examples.

### Example 1

After mixing 8 kg of stearic acid with 100 kg of zinc oxide, the mixture was stirred at room temperature to make it uniform. The mixture was heated to 115° C. and stirred for 1 hour. After the reaction, it was cooled to room temperature, crushed, and sieved through 200 mesh to produce zinc oxide coated with zinc-fatty acid soap.

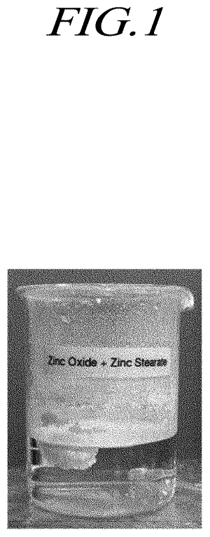

To confirm that zinc oxide was coated with zinc-fatty acid soap, a photo taken when it was put in water and boiled for 10 minutes is shown in FIG. 1. If the coating did not occur properly, the fatty acid would separate when heated, and the fatty acid would float on the surface of the water due to its specific gravity, and the zinc oxide would gather at the bottom of the water layer. As shown in FIG. 1, no zinc oxide fell to the bottom of the water side, indicating that stearic acid was bound to the surface of the zinc oxide.

### Example 2

Titanium dioxide of 100 kg and zinc oxide of 7 kg were mixed, stirred at room temperature to make it uniform. Stearic acid of 12 kg was mixed thereto, stirred at room temperature to make it uniform. This was heated to 115° C. and stirred for 1 hour. After the reaction, it was cooled to room temperature, crushed, and sieved through 150 mesh to produce titanium dioxide coated with fatty acids.

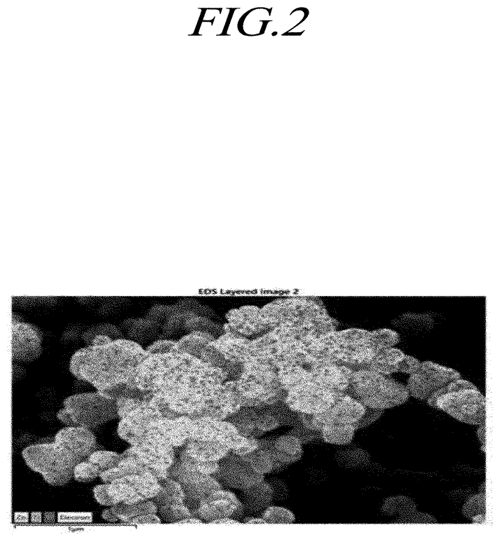

FIG. 2 is an energy dispersive spectroscopic analysis (EDS) photograph of a scanning electron microscope (SEM) of the powder of Example 2. It can be seen from FIG. 2 that zinc-stearic acid metal soap is uniformly distributed on the surface of the titanium dioxide.

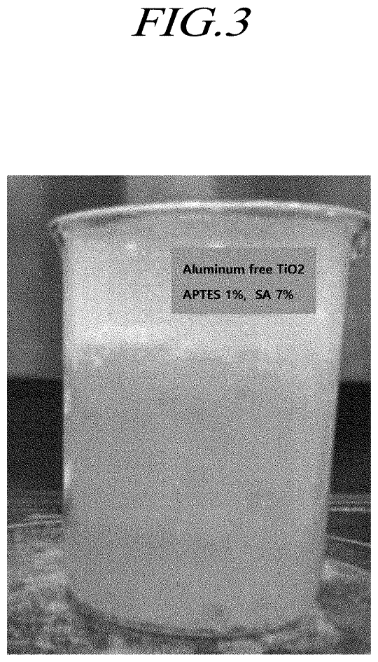

FIG. 3 is a photograph of titanium dioxide powder (Comparative Example 1) manufactured in the same manner as Example 2 except that zinc oxide was not mixed in Example 2, which was boiled in water for 1 minute. In FIG. 3, it can be seen that titanium dioxide and stearic acid are separated instantly.

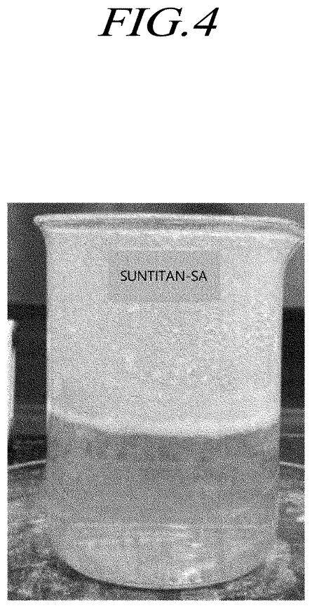

FIG. 4 is a photograph of titanium dioxide coated with stearic acid using aluminum hydroxide in a wet manner (Comparative Example 2) after boiling in water for 30 minutes. From FIG. 4, it can be seen that titanium dioxide is not separated, indicating that aluminum-stearic acid metal soap is chemically bonded and coated on the surface of titanium dioxide.

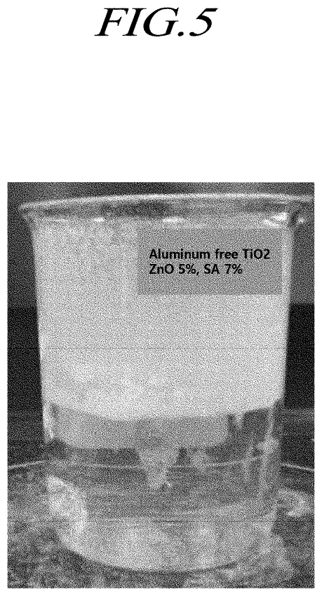

FIG. 5 is a photograph of titanium dioxide powder manufactured in Example 2 of the present invention after boiling in water for 30 minutes. From this photograph, it can be seen that titanium dioxide is not separated, indicating that zinc-stearic acid metal soap is chemically bonded and coated on the surface of titanium dioxide.

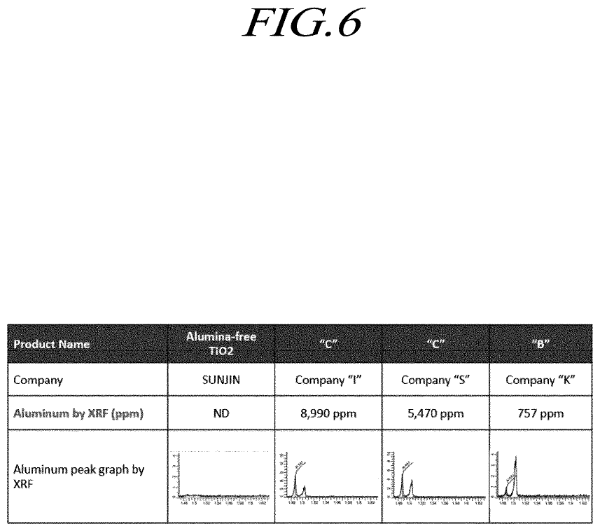

FIG. 6 shows the results of analysis using WD-XRF (Wavelength Dispersion X-ray Fluorescence Spectrometer) to confirm the presence of aluminum components in titanium dioxide powder including a zinc-fatty acid coating layer of Example 2 of the present invention and those of competitors (3 companies). As shown in FIG. 6, it can be confirmed that while the products of competitors have 8990 ppm, 5470 ppm, and 757 ppm, respectively, in the case of the present invention, no aluminum component is detected at all.

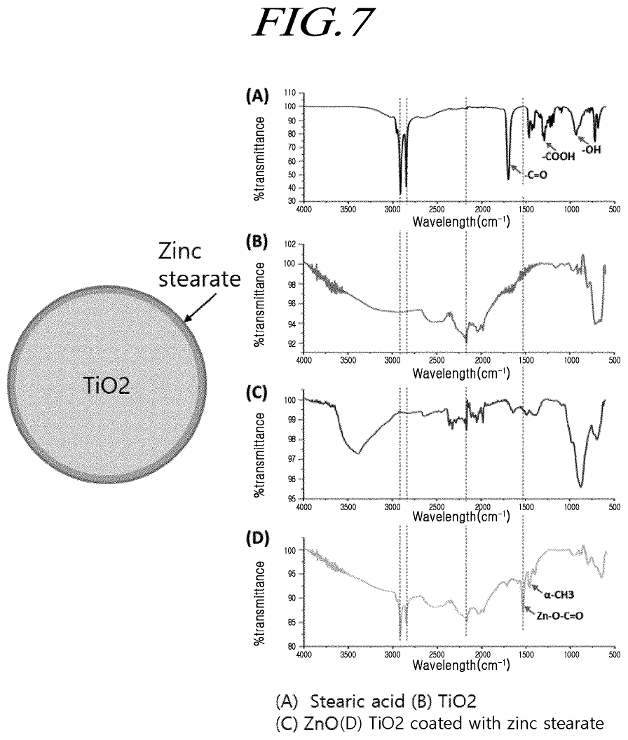

FIG. 7 is a result of analyzing the titanium dioxide powder including a zinc-fatty acid coating layer according to Example 2 of the present invention. As shown in FIG. 7, it can be clearly confirmed that the titanium dioxide powder of the present invention has a zinc-fatty acid coating layer formed thereon.

### Example 3

After mixing 100 kg of silica and 7 kg of zinc oxide, they were stirred at room temperature to make them uniform. After mixing 12 kg of isostearic acid, they were stirred at room temperature to make them uniform. They were heated to 120°1C. and stirred while reacting for 1 hour. After the reaction, they were cooled to room temperature, crushed, and sieved through 200 mesh to produce silica coated with zinc-fatty acid soap.

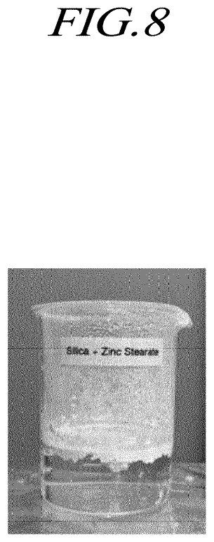

FIG. 8 is a photograph of the zinc-fatty acid soap-coated silica powder produced in Example 3 of the present invention when it was put into water and boiled for 10 minutes. As shown in FIG. 8, it can be seen that the silica is not separated from isostearic acid and did not precipitate to the bottom of the water, indicating that a solid coating of zinc-isostearic acid soap was formed on the surface of the silica.

### Comparative Example 3

After mixing 100 kg of silica and 10 kg of zinc hydroxide, they were stirred at room temperature to make them uniform. Here, 12 kg of isostearic acid was mixed and stirred at room temperature to make it uniform. This was heated to 120° C. and stirred for 1 hour. After the reaction, it was cooled to room temperature, crushed, and sieved through 200 mesh to produce silica coated with zinc-fatty acid soap.

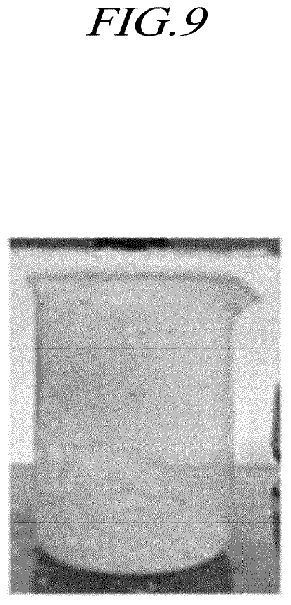

FIG. 9 is a photograph of the zinc-fatty acid soap-coated silica powder manufactured in Comparative Example 3 of the present invention when it was put into water and boiled for 10 minutes. In FIG. 9, it can be seen that isostearic acid was separated and silica was precipitated to the bottom of the water, indicating that zinc-isostearic acid soap did not form a solid coating on the surface of the silica.

### Example 4

Sericite of 100 kg and zinc oxide of 10 kg were mixed and stirred at room temperature to make it uniform. Palmitic acid of 15 kg was mixed here and stirred at room temperature to make it uniform. It was heated to 125° C. and stirred for 1 hour. After the reaction, it was cooled to room temperature, crushed, and sieved through 200 mesh to produce silica coated with zinc-fatty acid soap.

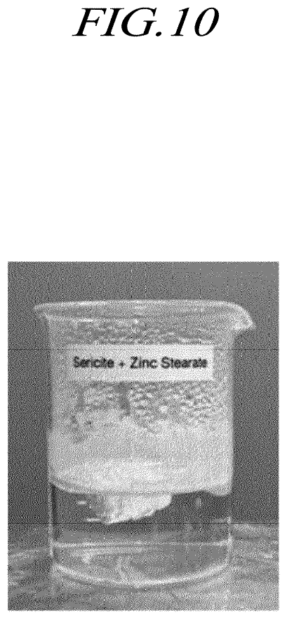

FIG. 10 is a photograph of the zinc-fatty acid soap-coated sericite powder produced in Example 4 of the present invention when it was put into water and boiled for 10 minutes. As shown in FIG. 10, it can be seen that the sericite was not separated from palmitic acid and does not settle to the bottom of the water, indicating that a solid coating of zinc-palmitic acid soap was formed on the surface of the sericite.

### Example 5

After mixing 100 kg of yellow iron oxide and 10 kg of zinc oxide, the mixture was stirred at room temperature to make it uniform. After mixing 14 kg of stearic acid, the mixture was stirred at room temperature to make it uniform. The mixture was heated to 120° C. and stirred for 1 hour. After the reaction, it was cooled to room temperature, crushed, and sieved through 200 mesh to produce yellow iron oxide coated with zinc-fatty acid soap.

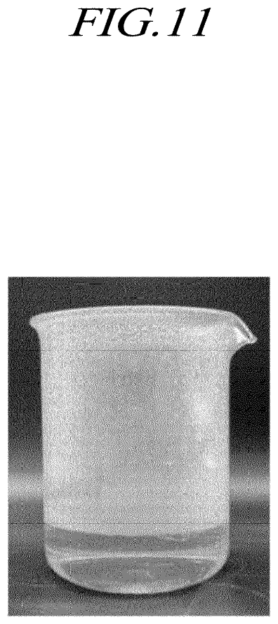

FIG. 11 is a photograph of the zinc-fatty acid soap-coated yellow iron oxide powder produced in Example 5 of the present invention when it was put into water and boiled for 10 minutes. As shown in FIG. 11, it can be seen that the yellow iron oxide does not separate from stearic acid and does not precipitate to the bottom of the water, indicating that a solid zinc-stearic acid soap coating was formed on the surface of the yellow iron oxide.

### Example 6

After mixing 100 kg of red iron oxide and 9 kg of zinc oxide, they were stirred at room temperature to make them uniform. Then, 12 kg of isostearic acid was mixed thereto, stirred at room temperature to make them uniform. It was heated to 115° C. and stirred while reacting for 1 hour. After the reaction, it was cooled to room temperature, crushed, and sieved through 200 mesh to produce red iron oxide coated with zinc-fatty acid soap.

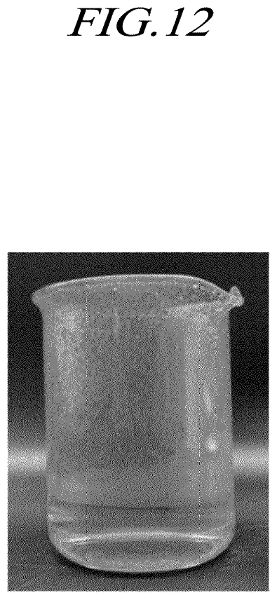

FIG. 12 is a photograph of the red iron oxide powder coated with zinc-fatty acid soap produced in Example 6 of the present invention when it was put into water and boiled for 10 minutes. As shown in FIG. 12, it can be seen that the red iron oxide is not separated from the isostearic acid and does not precipitate to the bottom of the water, indicating solid coating of zinc-isostearic acid soap was formed on the surface of the red iron oxide.

1. Inorganic powders for cosmetics coated with fatty acids, comprising:
a zinc-fatty acid soap coating layer on surface of the inorganic powders through dry coating.

2. Inorganic powders of claim 1, wherein the inorganic powders at least one selected from the group consisting of titanium dioxide, mica, talc, kaolin, sericite, yellow iron oxide, red iron oxide, black iron oxide, brown iron oxide, silica, zinc oxide and titanium dioxide mica.

3. Inorganic powders of claim 1, wherein the fatty acid is at least one selected from the group consisting of caprylic acid, capric acid, lauric acid, myristic acid, palmitic acid, stearic acid, isostearic acid, hydroxystearic acid, oleic acid, linoleic acid, and behenic acid.

4. Inorganic powders of claim 1, wherein the zinc-fatty acid soap coating layer is included in an amount of 2 to 50 parts by weight based on 100 parts by weight of the inorganic powders.

5. Inorganic powders of claim 1, wherein the inorganic powders include impurities including aluminum and the aluminum content is a trace amount of 20 ppm or less.

6. A method of preparing inorganic powders for cosmetics coated with zinc-fatty acid soap, comprising:
a first step of homogeneously mixing an inorganic powder and zinc oxide;
a second step of homogeneously stirring mixture of the first step by adding fatty acid;
a third step of heating and reacting stirred material of the second step; and
a fourth step of cooling and pulverizing reactant of the third step.

7. The method of preparing inorganic powders for cosmetics coated with zinc-fatty acid soap of claim 6, wherein in the first step, 1 to 20 parts by weight of zinc oxide is included relative to 100 parts by weight of the inorganic powders.

8. The method of preparing inorganic powders for cosmetics coated with zinc-fatty acid soap of claim 6, wherein in the second step, 50 to 500 parts by weight of fatty acid is included relative to 100 parts by weight of the zinc oxide.

9. The method of preparing inorganic powders for cosmetics coated with zinc-fatty acid soap of claim 6, wherein in that in the third step, heating is performed at 100 to 150°C.

10. The method of preparing inorganic powders for cosmetics coated with zinc-fatty acid soap of claim 6, wherein the inorganic powders include impurities including aluminum and the aluminum content is a trace amount of 20 ppm or less.

11. A cosmetic comprising inorganic powders of claim 1.
